(19) United States
(12) Patent Application Publication
(10) Pub. No.: US 2025/0194937 A1
(43) Pub. Date: Jun. 19, 2025

(54) PHYSIOLOGIC SENSORS TO AUGMENT PVC BURDEN INFORMATION

(71) Applicant: Cardiac Pacemakers, Inc., St. Paul, MN (US)

(72) Inventors: Pramodsingh Hirasingh Thakur, Woodbury, MN (US); David L. Perschbacher, Blaine, MN (US)

(21) Appl. No.: 18/968,462

(22) Filed: Dec. 4, 2024

Related U.S. Application Data
(60) Provisional application No. 63/611,623, filed on Dec. 18, 2023.

(51) Int. Cl. A61B 5/02; A61B 5/00; A61B 5/332; A61B 5/346; A61B 7/04; A61B 8/08

(52) U.S. Cl. CPC A61B 5/02028; A61B 5/332; A61B 5/346; A61B 5/746; A61B 7/04; A61B 8/0883

(57) ABSTRACT

Systems and methods are disclosed to a medical device system comprising a cardiac signal sensing circuit, a heart sound sensor, a signal processing circuit, and a control circuit. The cardiac signal sensing circuit produces a sensed cardiac signal representative of cardiac depolarization signals when connected to electrodes. The heart sound sensor produces a sensed heart sound signal representative of vibrational sounds of the heart. The signal processing circuit identifies premature ventricular contractions (PVCs) using sensed cardiac signal information. The control circuit is configured to calculate a PVC burden of the patient using PVC information, determine that the calculated PVC burden exceeds a threshold PVC burden and that the patient is asymptomatic for heart failure, determine that sensed heart sound signal information indicates heart failure of the patient, and present a recommendation of an echocardiogram for the patient to a user.

### CLAIM OF PRIORITY

This application claims the benefit of U.S. Provisional Application No. 63/611,623, filed on Dec. 18, 2023, which is hereby incorporated by reference in its entirety.

### TECHNICAL FIELD

This document relates generally to medical devices and more particularly to systems, methods, and devices to estimate conducted premature ventricular contraction burden of a patient.

### BACKGROUND

Ambulatory medical devices (AMDs), including implantable, subcutaneous, insertable, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Early correct identification and management of the risk of the worsening condition of the patient may avoid unnecessary medical interventions, extend the usable life of ambulatory medical devices, and reduce healthcare costs.

### SUMMARY

Systems and methods are disclosed to monitor the heart failure status of patients through monitoring conducted premature ventricular contractions (PVCs) augmented with information from other physiologic sensors. PVCs may be a predictor of future reduction in left ventricular ejection fraction (LVEF).

In a first Example (Example 1) a medical device system includes a cardiac signal sensing circuit, a heart sound sensor, a signal processing circuit, and a control circuit. The cardiac signal sensing circuit produces a sensed cardiac signal representative of cardiac depolarization signals when connected to electrodes. The heart sound sensor produces a sensed heart sound signal representative of vibrational sounds of the heart. The signal processing circuit identifies premature ventricular contractions (PVCs) using sensed cardiac signal information. The control circuit is configured to calculate a PVC burden of the patient using PVC information, determine that the calculated PVC burden exceeds a threshold PVC burden and that the patient is asymptomatic for heart failure, determine that sensed heart sound signal information indicates heart failure of the patient, and present a recommendation of an echocardiogram for the patient to a user.

In Example 2, the subject matter of Example 1 optionally includes a control circuit configured to determine an S1 heart sound magnitude using the sensed heart sound signal information, compare the determined S1 heart sound magnitude to a baseline S1 heart sound magnitude of the patient, detect a decrease in the determined S1 heart sound magnitude from the baseline S1 heart sound magnitude, and generate the recommendation of the echocardiogram in response to the calculated PVC burden exceeding the threshold PVC burden and the detected decrease in the S1 heart sound magnitude.

In Example 3, the subject matter of one or both of Examples 1 and 2 optionally include a control circuit configured to compare the sensed heart sound signal information to a baseline heart sound signal for the patient, detect a change in the sensed heart sound signal from the baseline heart sound signal that includes an S3 heart sound, and generate the recommendation of the echocardiogram in response to the calculated PVC burden exceeding the threshold PVC burden and the detecting the S3 heart sound.

In Example 4, the subject matter of one or any combination of Examples 1-3 optionally includes a control circuit configured to receive information of left ventricle ejection fraction (LVEF) of the patient, and generate the recommendation of the echocardiogram in response to the calculated PVC burden exceeding the threshold PVC burden, the LVEF information indicating normal LVEF, and sensed heart sound signal indicating heart failure.

In Example 5, the subject matter of one or any combination of Examples 1-4 optionally include a control circuit configured to receive information of physiologic symptoms of the patient associated with heart failure, and present an alert of heart failure status of the patient to the user when determining the patient is symptomatic for heart failure and the PVC burden exceeds the threshold PVC burden.

In Example 6, the subject matter of one or any combination of Examples 1-5 optionally includes an ambulatory medical device (AMD) and a second device. The AMD includes the cardiac signal sensing circuit, the heart sound signal sensing circuit, and a first communication circuit configured to communicate information with the second device. The second device includes the signal processing circuit, the control circuit, and a second communication circuit configured to communicate information with the AMD, and the control circuit is configured to upload the sensed cardiac signal information and the sensed heart sound signal information from the AMD.

In Example 7, the subject matter of one or any combination of Examples 1-5 optionally includes an ambulatory medical device (AMD) and a second device. The AMD includes the cardiac signal sensing circuit, the heart sound signal sensing circuit, and a first communication circuit configured to communicate information with the second device. The second device includes the signal processing circuit, the control circuit, and a second communication circuit configured to communicate information with the AMD. The control circuit is configured to upload the sensed cardiac signal information from the AMD, send a command to enable the AMD to produce the sensed heart sound signal in response to determining that the calculated PVC burden exceeds the threshold PVC burden and that the patient is asymptomatic for heart fail, and upload the sensed heart sound signal information from the AMD.

In Example 8, the subject matter of one or any combination of Examples 1-5 optionally includes an ambulatory medical device (AMD) and a second device. The AMD includes the cardiac signal sensing circuit, the heart sound signal sensing circuit, the signal processing circuit, and a first communication circuit configured to communicate information with the second device. The second device includes the control circuit, and a second communication circuit configured to communicate information with the AMD. The control circuit is configured to upload the PVC information and the sensed heart sound signal information from the AMD.

Example 9 includes subject matter (such as a method of operating a medical device system) or can optionally be combined with one or any combination of Examples 1-8 to include such subject matter, comprising sensing a cardiac signal of a patient using an ambulatory medical device (AMD) of the medical device system, identifying premature ventricular contractions (PVCs) in the sensed cardiac signal, computing a PVC burden of the patient, determining that the patient is asymptomatic for heart failure and the computed PVC burden exceeds a threshold PVC burden, sensing a heart sound signal of the patient using the AMD, determining that the heart sound signal indicates heart failure of the patient, and generating a recommendation to a user of further screening of the patient for heart failure.

In Example 10, the subject matter of Example 9 optionally includes determining a baseline S1 heart sound magnitude of the patient, and detecting a decrease in S1 heart sound magnitude in the sensed heart sound signal from the baseline heart sound magnitude.

In Example 11, the subject matter of one or both of Examples 9 and 10 optionally includes detecting an S3 heart sound in the heart sound signal.

In Example 12, the subject matter of one or any combination of Examples 9-11 optionally includes sensing the heart sound signal when the patient has normal left ventricle ejection fraction (LVEF) and the computed PVC burden exceeds the threshold PVC burden.

In Example 13, the subject matter of one or any combination of Examples 9-12 optionally includes determining the patient is symptomatic for heart failure and the PVC burden exceeds the threshold PVC burden, and generating an alert of heart failure status of the patient.

In Example 14, the subject matter of Example 13 optionally includes receiving information of physiologic symptoms associated with heart failure reported for the patient.

In Example 15, the subject matter of one or any combination of Examples 9-14 optionally includes uploading the sensed cardiac signal from the AMD to a second device of the medical device system according to a schedule and wherein the identifying the PVCs includes the second device identifying PVCs in the uploaded cardiac signals and computing the PVC burden, sending, by the second device, a command to the AMD to sense the heart sound signal in response to the computed PVC burden exceeding the threshold PVC burden, and uploading the sensed heart signal to the second device, and wherein the determining that the heart sound signal indicates heart failure includes the second device determining that the heart sound signal indicates heart failure.

Example 16 includes subject matter (such as a medical device system) or can optionally be combined with one or any combination of Examples 1-15 to include such subject matter, comprising a signal receiver circuit and a control circuit. The signal receiver circuit is configured to receive cardiac depolarization information of a patient and heart sound information of the patient. The control circuit is configured to identify premature ventricular contractions (PVCs) using the cardiac depolarization information, calculate a PVC burden of the patient, determine that the calculated PVC burden exceeds a threshold PVC burden and that the patient is asymptomatic for heart failure, determine that the received heart sound information indicates heart failure of the patient, and present a recommendation of an echocardiogram for the patient to a user.

In Example 17, the subject matter of Example 16 optionally includes a storage device storing a database that includes information of reported patient symptoms, and a control circuit configured to determine that the patient is asymptomatic using the information of reported patient symptoms.

In Example 18, the subject matter of Example 17 optionally includes a control circuit configured to determine that the patient is symptomatic for heart failure using the information of reported patient symptoms, and present an alert of heart failure status of the patient to the user when determining the patient is symptomatic for heart failure and the PVC burden exceeds the threshold PVC burden.

In Example 19, the subject matter of Example 16 optionally includes a storage device storing a database that includes patient information including a baseline S1 heart sound magnitude of the patient, and a control circuit configured to determine an S1 heart sound magnitude using the received heart sound information, compare the determined S1 heart sound magnitude to the baseline S1 heart sound magnitude, detect a decrease in the determined S1 heart sound magnitude from the baseline S1 heart sound magnitude, and generate the recommendation of the echocardiogram in response to the calculated PVC burden exceeding the threshold PVC burden and the detected decrease in the S1 heart sound magnitude.

In Example 20, the subject matter of Example 16 optionally includes a storage device storing a database that includes patient information including a baseline heart sound signal for the patient and a control circuit configured to compare the received heart sound information to baseline heart sound signal, detect a change in the received heart sound information from the baseline heart sound signal that includes an S3 heart sound, and generate the recommendation of the echocardiogram in response to the calculated PVC burden exceeding the threshold PVC burden and the detecting the S3 heart sound.

The Examples can be combined in any permutation or combination.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a patient management system.

FIG. 2 illustrates an example of an ambulatory medical device (AMD) electrically coupled to a heart.

FIG. 3 is a block diagram of portions of an example of a medical device system.

FIG. 4 is a flow diagram of a method of operating a medical device system.

FIG. 5 is a block diagram of portions of an example of a medical device system.

FIG. 6 is a block diagram of an example machine upon which any one or more of the techniques (e.g., methodologies) discussed herein may perform.

### DETAILED DESCRIPTION

Ambulatory medical devices can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

A sinus node located in the atria typically initiates cardiac contractions of a patient by regularly occurring sinus impulses that cause atrial depolarization followed by ventricular depolarization. A premature ventricular contraction (PVC) can occur in a patient when Purkinje fibers initiate a depolarization before a sinus impulse. PVCs may be a predictor of future reduction in left ventricular ejection fraction (LVEF) and worsening of heart failure status of a patient.

PVC burden is measure of the frequency that a patient experiences PVCs. PVC burden can be calculated as the percentage of the heart beats of the patient that have a PVC. Patients with an elevated PVC burden (e.g., greater than 5%) can be screened using an annual echocardiogram to determine their heart failure status. More frequent screening is usually not performed because of the expense associated with the echocardiogram. For patients that are truly at risk of cardiomyopathy, annual screening may delay detection of the patient's condition. Device-based monitoring of the patient's condition can provide early identification of patients that would benefit from timely screening for worsening heart failure.

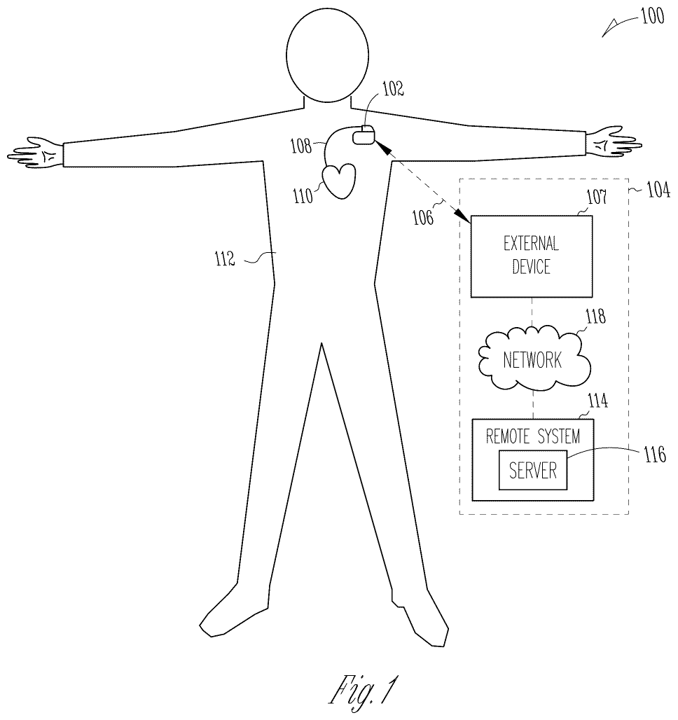

FIG. 1 illustrates portions of an example of a patient management system and portions of an environment in which the system 100 can be used. The system 100 can include an AMD 102 that is implantable, an external system 104, and a communication link such as a telemetry link 106. The AMD 102 can include an electronic unit coupled by a cardiac lead 108, or additional leads, to a heart 110 of a subject 112. Examples of the AMD 102 can include, but are not limited to, pacemakers, pacemaker/defibrillators, cardiac resynchronization devices, cardiac remodeling control devices, and insertable cardiac monitors (ICMs). In an example, the AMD 102 can be configured to monitor health of the heart 110 and determine one or more abnormalities associated with the heart 110. The AMD 102 can take a necessary action, such as stimulating one or more portions of the heart 110 through the lead 108, to treat the one or more abnormalities.

In an example, the external system 104 can include an external device 107 configured to communicate bi-directionally with the AMD 102 such as through the telemetry link 106. For example, the external device 107 can include a programmer to program the AMD 102 to provide one or more therapies to the heart 110. In an example, the external device 107 can program the AMD 102 to detect presence of a conduction block in a left bundle branch (LBB) of the heart 110 and prevent dyssynchronous contraction of the heart 110 by providing a cardiac resynchronization therapy (CRT) to the heart 110.

In an example, the external device 107 can be configured to transmit data to the AMD 102 through the telemetry link 106. Examples of such transmitted data can include programming instructions for the AMD 102 to acquire physiological data, perform at least one self-diagnostic test (such as for a device operational status), or deliver at least one therapy or any other data. In an example, the AMD 102 can be configured to transmit data to the external device 107 through the telemetry link 106. This transmitted data can include real-time physiological data acquired by the AMD 102 or stored in the AMD 102, therapy history data, an operational status of the AMD 102 (e.g., battery status or lead impedance), and the like. The telemetry link 106 can include an inductive telemetry link or a far-field radio-frequency telemetry link.

In an example, the external device 107 can be a part of a system 100 that can include other devices such as a remote system 114 for remotely programming the AMD 102. In an example, the remote system 114 can be configured to include a server 116 that can communicate with the external device 107 through a telecommunication network 118 such as to access the AMD 102 to remotely monitor the health of the heart 110 or adjust parameters associated with the one or more therapies.

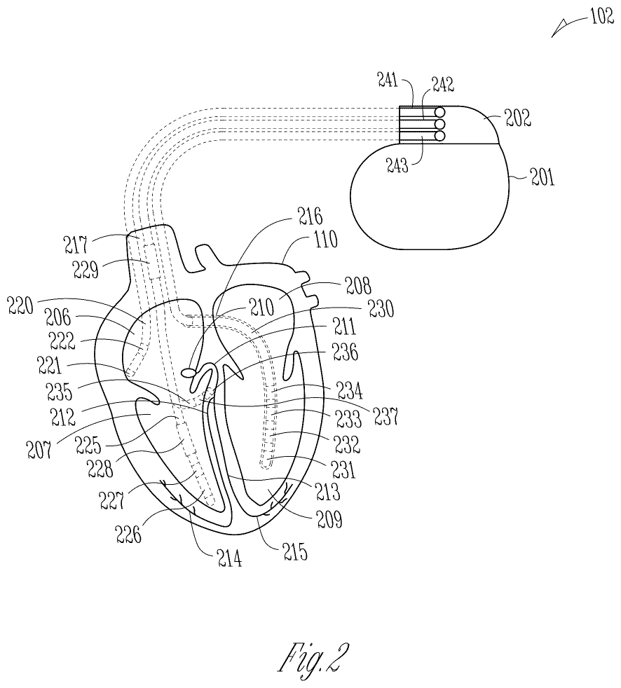

FIG. 2 illustrates an AMD 102 that is an implantable medical device (IMD). The AMD 102 is electrically coupled to a heart 110, such as through one or more leads coupled to the AMD 102 through one or more lead ports, such as first, second, or third lead ports 241, 242, 243 in a header 202 of the AMD 102. In an example, the AMD 102 can include an antenna, such as in the header 202, configured to enable communication with an external system and one or more electronic circuits in a hermetically sealed housing (CAN) 201. The AMD 102 illustrates an example medical device (or a medical device system) as described herein.

The AMD 102 may include an implantable medical device (IMD), such as an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the AMD 102 can include one or more electrodes or other sensors (e.g., a pressure sensor, an accelerometer, a gyroscope, a microphone, etc.) powered by a power source in the AMD 102. The one or more electrodes or other sensors of the leads, the AMD 102, or a combination thereof, can be configured detect physiologic information from, or provide one or more therapies or stimulation to, the patient.

The AMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN 201 such as for unipolar sensing of an electrogram or for delivering one or more pacing pulses. A defibrillation electrode (e.g., the first defibrillation coil electrode 228, the second defibrillation coil electrode 229, etc.) may be used together with the CAN 201 to deliver one or more cardioversion/defibrillation pulses.

In an example, the AMD 102 can sense impedance such as between electrodes located on one or more of the leads or the CAN 201. The AMD 102 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the AMD 102 can be configured to inject current between an electrode on one or more of the first, second, third, or fourth leads 220, 225, 230, 235 and the CAN 201, and to sense the resultant voltage between the same or different electrodes and the CAN 201.

The example lead configurations in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively, and a fourth lead 235 positioned in the RV 207 near the His bundle 211, between the AV node 210 and the right and left bundle branches 212, 213 and Purkinje fibers 214, 215. Each lead can be configured to position one or more electrodes or other sensors at various locations in or near the heart 110 to detect physiologic information or provide one or more therapies or stimulation.

The first lead 220, positioned in the RA 206, includes a first tip electrode 221 located at or near the distal end of the first lead 220 and a first ring electrode 222 located near the first tip electrode 221. The second lead 225 (dashed), positioned in the RV 207, includes a second tip electrode 226 located at or near the distal end of the second lead 225 and a second ring electrode 227 located near the second tip electrode 226. The third lead 230, positioned in the coronary vein 216 over the LV 209, includes a third tip electrode 231 located at or near the distal end of the third lead 230, a third ring electrode 232 located near the third tip electrode 231, and two additional electrodes 233, 234. The fourth lead 235, positioned in the RV 207 near the His bundle 211, includes a fourth tip electrode 236 located at or near the distal end of the fourth lead 235 and a fourth ring electrode 237 located near the fourth tip electrode 236. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 includes a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 211.

The AMD 102 can include a heart sound sensor to produce a heart sound signal. Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4).

Valve closures and blood movement and pressure changes in the heart can cause accelerations, vibrations, or movement of the cardiac walls that can be detected using a heart sound sensor such as an accelerometer or a microphone, producing a heart sound signal. In an example, heart sound signal portions, or values of respective heart sound signals for a cardiac interval, may be detected by comparison with a sensed cardiac signal. For instance, the value and timing of an S1 signal can be detected using an amplitude or energy of the heart sound signal occurring at or about the R wave of the cardiac interval. The S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles. For example, a heart sound parameter can include a composite S1 parameter representative of a plurality of S1 parameters, for example, over a certain time period (e.g., a number of cardiac cycles, a representative time period, etc.). In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform.

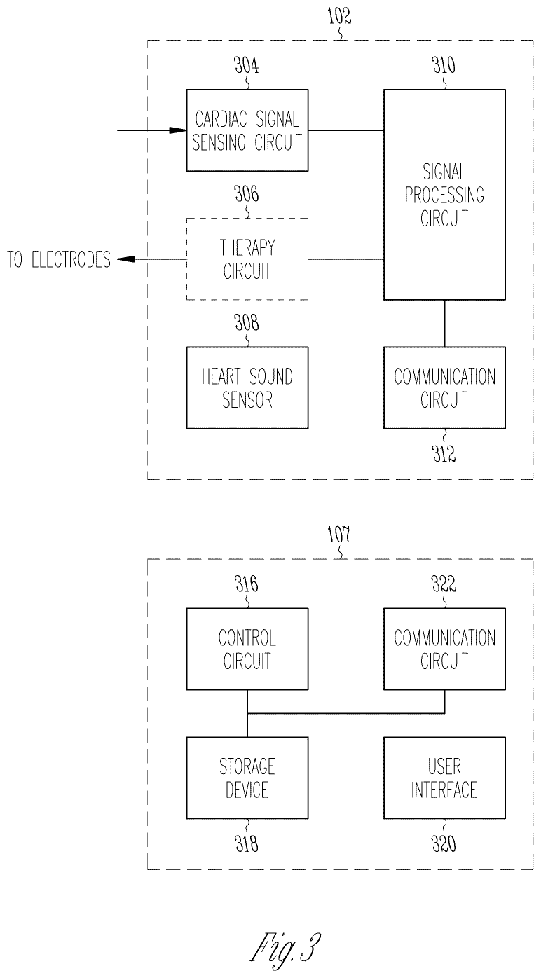

FIG. 3 is a block diagram of portions of electronic circuits of an example of a medical device system to monitor heart failure status of a patient. The system 300 includes a cardiac signal sensing circuit 304, a heart sound sensor 308, a signal processing circuit 310, and a control circuit 316. The cardiac signal circuit 304 is configured to produce a sensed cardiac signal representative of electrical depolarization signals of a heart of a patient when connected to electrodes (e.g., any of the electrodes in FIG. 2). The cardiac signal sensing circuit 304 includes one or more sense amplifiers to sense one or both of a voltage signal or a current signal at the electrodes. Cardiac electrical information of the patient can be sensed using the cardiac signal sensing circuit 304. Timing metrics between different features in a sensed electrical signal (e.g., first and second cardiac features, etc.) can be determined, such as by signal processing circuitry 310 or the control circuit 316. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

The heart sound sensor is configured to produce a sensed heart sound signal representative of vibrational sounds of the heart, such as any of the S1, S2, S3, and S4 heart sounds. The control circuit 316 may be implemented using an application-specific integrated circuit (ASIC) constructed to perform one or more functions or a general-purpose circuit programmed to perform the functions. A general-purpose circuit can include, among other things, a microprocessor or a portion thereof, a microcontroller or a portion thereof, and a programmable logic circuit or a portion thereof. The storage device 318 may be a memory integral to the programming control circuit 316, or a separate memory device. The signal processing circuit 310 processes signals produced by the cardiac signal sensing circuit 304 and the heart sound sensor 308. The signal processing circuit 310 may also be implemented using an ASIC constructed to perform one or more functions or a general-purpose circuit programmed to perform the functions.

The electronic circuits of a medical device system may be included in more than one device. For example, as shown by the dashed lines in FIG. 3, the cardiac signal sensing circuit 304, the heart sound sensor 308, and the signal processing circuit 310 may be included in an AMD 102 (e.g., AMD 102 in FIG. 1 or FIG. 2). The AMD 102 may be implantable and may be coupled to multiple implantable electrodes, such as the electrode arrangement described in the example of FIG. 2. The AMD 102 may include a therapy circuit 306 to provide electrical pacing therapy or defibrillation therapy to the patient when operatively coupled to electrodes, or the AMD 102 can be a diagnostic-only device and not provide therapy to the patient.

The control circuit 316 may be included in an external device 107 (e.g., external device 107 in FIG. 1). The AMD 102 and the external device 107 communication information using communications circuits 312 and 322. The information is communicated wirelessly using near-field inductive wireless signals or far-field radio-frequency signals. The external device 107 includes a user interface 320 to present information to a user and to receive prompts to perform actions selected by the user. In variations, the signal processing circuit 310 is included in the external device 107 and the external device 107 performs the signal processing on cardiac signals and heart sound signals sensed by the AMD 102.

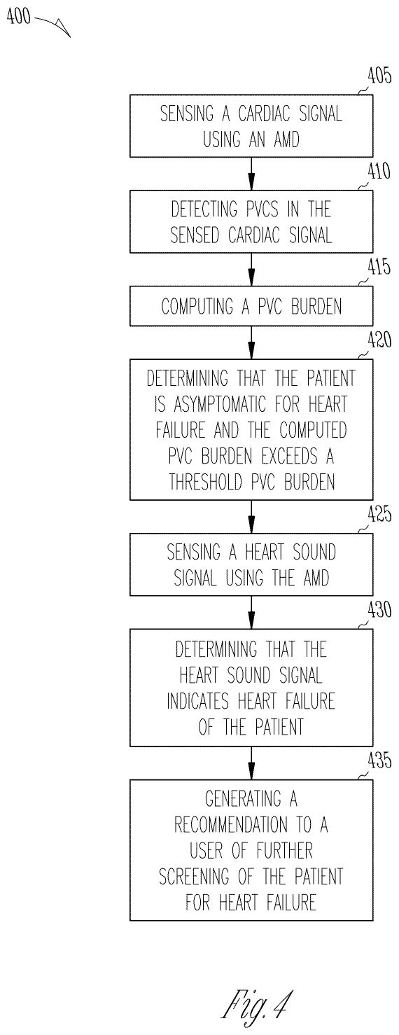

FIG. 4 is a flow diagram of an example of a method 400 of operating a medical device system (e.g., the medical device system 300 of FIG. 3). The method 400 identifies patients that would benefit from further screening for worsening heart failure. The patients are identified by determining PVC burden and augmenting the PVC burden using device-based physiological sensors. The early identification and further screening can result in early detection of worsening of heart failure status of the patients.

At block 405, a cardiac signal of a patient is sensed using a cardiac signal sensing circuit 304 of the medical device system 300. At block 410, PVCs are identified in cardiac signals sensed using the AMD. To identify a PVC, in some examples the signal processing circuit 310 labels or otherwise identifies two ventricular depolarizations that occur, without an intervening atrial depolarization or beat. The signal processing circuit 310 may provide an indication that a PVC has occurred when detecting less than two atrial beats during two ventricular depolarizations. In another example of detecting a PVC, the signal processing circuit 310 may identify that a PVC occurred when detecting an atrio-ventricular (AV) interval that is less than a specified threshold AV interval. These detected events may indicate ventricular events that are not driven by the atria.

At block 415, a PVC burden is computed or calculated for the patient. The PVC burden may be calculated by the control circuit 316 of the system 300 or the signal processing circuit 310 of the system. The PVC burden may be calculated as a percentage of the total heart beats with a PVC detected or calculated as a ratio including the total number of heart beats and the number of heart beats with PVCs.

At block 420, the control circuit 316 determines if the PVC burden exceeds a specified PVC burden threshold (e.g., whether the calculated PVC burden is greater than 5%). If the PVC burden is greater than the threshold, the control circuit determines whether the patient is asymptomatic for heart failure. In some examples, the storage device 318 stores a database that includes information of reported patient symptoms. The patient may report symptoms using a personal device (e.g., an application or “app” of a smartphone) and the reported symptoms are added to the data base. The patient may be asked to report symptoms associated with heart failure, such as if the patient experiences heart flutter, heart palpitations, dizziness or near-fainting, a pounding sensation in the chest, feeling of pressure in the neck, fatigue, etc. The control circuit 316 may interpret the patient as being asymptomatic due to no reported symptoms in the stored database.

At block 425, a heart sound signal is sensed using the heart sound sensor 308 of the system 300. In some examples, heart sound signals are sensed with the cardiac signals and sensed heart sound signal information is provided to the control circuit 316. The signal processing circuit 310 may process sensed heart sound signals to produce the sensed heart sound signal information provided to the control circuit 316. In some examples, the heart sound signal is sensed in response to a command from the control circuit 316. The control circuit 316 may send the command to the AMD to enable the heart sound sensor when the PVC burden is greater than the threshold and the patient is asymptomatic for heart failure. The heart sound information is later uploaded from the AMD. Using triggered heart sound sensing instead of constant heart sound sensing can reduce drain on the battery of AMD.

At block 430, the control circuit determines that the sensed heart sound information indicates heart failure of the patient. In some examples, the sensed heart sound information includes information of sensed S1 heart sounds. The S1 heart sound information may indicate heart failure when indicating that the magnitude of the S1 heart sound is decreased. The S1 heart sound can be a proxy measure for contractile function of the ventricles. A reduction in S1 magnitude may indicate a reduction in contractile function of the ventricles. In some examples, a baseline S1 heart sound magnitude is determined and stored in the storage device 318. The control circuit determines the S1 heart sound magnitude using the sensed heart sound signal information and compares the determined S1 heart sound magnitude to the patient's baseline S1 heart sound magnitude. If the current S1 magnitude has decreased from the baseline S1 magnitude by more than a threshold change in magnitude, the control circuit 316 may interpret the decrease as indicating heart failure.

In some examples, the sensed heart sound signal information includes information of sensed S3 heart sounds. Presence of S3 in the heart sound signal of the patient may be a specific marker of HF. A baseline heart sound signal can be sampled and stored in the storage device 318. The control circuit 316 compares the sensed heat sound signal information to a baseline heart sound signal. If the current sensed heart sound signal includes a change in S3 heart sound from the baseline heart sound signal, the control circuit 316 may interpret the change as indicating heart failure. Such a change may include an appearance of an S3 heart sound that wasn't present in the baseline heart sound signal, a significant increase in the magnitude of the S3 heart sound, or an increase in the ratio of the magnitude of the S3 heart sound to the magnitude of another sensed heart sound.

At 435, the control circuit 316 presents a recommendation to the user of further clinical screening of the patient for heart failure in response to the patient being asymptomatic for heart failure, the PVC burden exceeding the PVC burden threshold, and the heart sound signal information indicating heart failure. The recommendation may be presented on a display of the user interface 320. The recommendation of further screening may include a recommendation of scheduling an echocardiogram for the patient.

In some examples, the storage device includes information of left ventricle ejection fraction (LVEF) of the patient. The control circuit 316 may present a recommendation to the user of further screening of the patient for heart failure in response to the patient having normal LVEF, the PVC burden exceeding the PVC burden threshold, and the heart sound signal information indicating heart failure.

If the control circuit determines that the patient is symptomatic, the control circuit may heighten the recommendation to an alert. The control circuit 316 may interpret the patient as being symptomatic based on one or both of the number of reported symptoms and the types of reported symptoms in the stored database. The control circuit 316 present an alert of heart failure status of the patient to the user when determining the patient is symptomatic for heart failure and determining the PVC burden exceeds the threshold PVC burden.

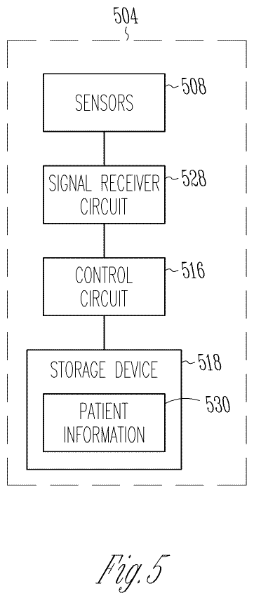

FIG. 5 is a block diagram of portions of electronic circuits of another example of a medical device system to monitor heart failure status of a patient. The system 504 includes a signal receiver circuit 528, a control circuit 516, and a storage device 518. The signal receiver circuit 528 receives cardiac depolarization information of a patient. In some examples, the system 504 is an external system and the signal receiver circuit 528 receives a cardiac depolarization signal from a separate device (e.g., an AMD). In some examples, the signal receiver circuit 528 receives cardiac depolarization information processed by the separate device. In some examples, the system 504 includes sensors 508 such as a cardiac signal sensing circuit 304 including sense amplifiers.

The signal receiver circuit 528 also receives heart sound information of a patient. In some examples, the signal receiver circuit 528 receives a heart sound signal from a separated device (e.g., an AMD). In some examples, the signal receiver circuit 528 receives heart sound information processed by the separate device. In some examples, the system 504 includes a heart sound sensor 308. In some examples, the sensors 508 include an accelerometer and the signal receiver circuit 528 is configured to process accelerometer signals to determine the heart sound information.

The storage device 518 stores patient information 530. The patient information 530 may include a database storing patient symptom information. In some examples, the patient information is stored in a cloud-based server, and the system 504 retrieves the patient information from the server. The control circuit 516 may be implemented using an application-specific integrated circuit (ASIC) constructed to perform one or more functions or a general-purpose circuit programmed to perform the functions. The control circuit 516 is configured to identify PVCs using the cardiac depolarization information received by the signal receiver circuit 528 and calculate a PVC burden of the patient.

The control circuit 516 compares the calculated PVC burden to a threshold PVC burden. When the calculated PVC burden exceeds the threshold PVC burden, the control circuit 516 determines from the stored patient information 530 whether the patient is asymptomatic for heart failure. If the control circuit 516 determines that the calculated PVC burden exceeds a threshold PVC burden and that the patient is asymptomatic for heart failure, the control circuit 516 determines whether the heart sound information indicates the patient has heart failure.

In some examples, the storage device 518 stores a baseline S1 heart sound magnitude of the patient. The control circuit 516 may compare the S1 heart sound magnitude of the received heart sound information to the baseline heart sound magnitude stored for the patient. The control circuit 516 may determine that the heart sound information indicates heart failure when the current S1 magnitude is less than the baseline S1 magnitude by more than a threshold magnitude difference.

In some examples, the storage device 518 stores a baseline heart sound signal with the patient information. Multiple heart sound signals may have recorded for the patient and averaged or otherwise filtered into the baseline heart sound signal. The control circuit 516 compares the received heart sound information to the baseline heart sound signal. The control circuit 516 may determine that the heart sound information indicates heart failure when detecting a change in the S3 heart sound of the received heart sound information from the baseline heart sound signal. For instance, the control circuit 516 may determine that the heart sound information indicates heart failure when detecting an S3 heart sound that is not present in the baseline heart sound signal. In another example, the control circuit 516 may determine that the heart sound information indicates heart failure when detecting an increase in the magnitude of the S3 heart sound from the baseline heart sound signal that is greater than a threshold magnitude difference.

If the control circuit 516 determines that the calculated PVC burden exceeds the threshold PVC burden, the patient is asymptomatic for heart failure, and the heart sound information indicates the patient has heart failure, the control circuit 516 generates a recommendation of further clinical screening for heart failure for the patient. In some examples, the system 504 includes a user interface 320, and the system 504 presents a recommendation of an echocardiogram for the patient to a user. In some examples, the system 504 sends the recommendation to the cloud-based server, and a different device is used to notify one or both of a clinician and the patient of the recommendation.

In some examples, the control circuit 516 determines that the calculated PVC burden exceeds the threshold PVC burden and that the patient is symptomatic for heart failure from the stored patient information 530. In this case, the control circuit 516 may generate and alert of the heart failure status of the patient. The system 504 may present the alert to the user or send the alert to the cloud-based server.

The techniques described herein allow for early identification of patients that are truly at risk of cardiomyopathy. The early device-based detection can identify those patients more likely to benefit from early clinical screening. The early identification results in better use of limited health care resources and allows for patients at risk to receive a timely diagnosis.

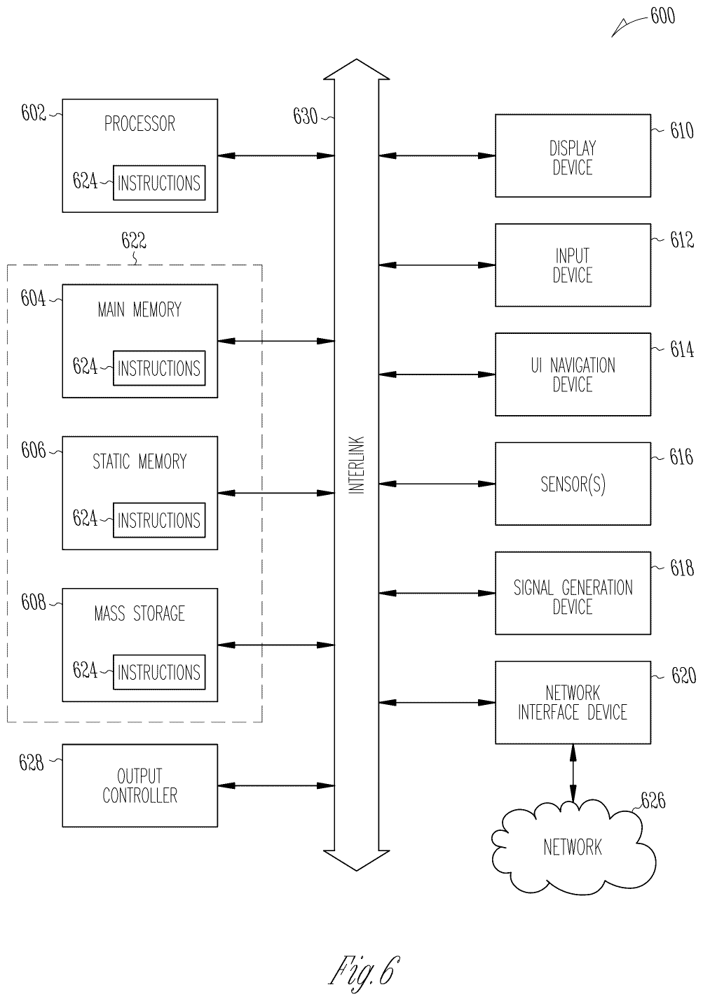

FIG. 6 is a block diagram of an example machine 600 upon which any one or more of the techniques (e.g., methodologies) discussed herein may perform. Portions of this description may apply to the computing framework of one or more of the medical devices described herein, such as the ambulatory medical device, the wearable medical device, the external programmer, the remoter server, etc., in computing a composite health index. Further, as described herein with respect to medical device components, systems, or machines, such may require regulatory-compliance not capable by generic computers, components, or machinery.

Examples, as described herein, may include, or may operate by, logic or a number of components, or mechanisms in the machine 600. Circuitry (e.g., signal processing circuitry, etc.) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 600 (e.g., computer system) may include a hardware processor 602 (e.g., a central processing unit (CPU), a graphics processing unit (GPU), a hardware processor core, or any combination thereof), a main memory 604, a static memory 606 (e.g., memory or storage for firmware, microcode, a basic-input-output (BIOS), unified extensible firmware interface (UEFI), etc.), and mass storage 608 (e.g., hard drive, tape drive, flash storage, or other block devices) some or all of which may communicate with each other via an interlink 630 (e.g., bus). The machine 600 may further include a display unit 610, an input device 612 (e.g., a keyboard), and a user interface (UI) navigation device 614 (e.g., a mouse). In an example, the display unit 610, input device 612, and UI navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, microphone, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 602, the main memory 604, the static memory 606, or the mass storage 608 may be, or include, a machine-readable medium 622 on which is stored one or more sets of data structures or instructions 624 (e.g., software) embodying or utilized by any one or more of the techniques or functions described herein. The instructions 624 may also reside, completely or at least partially, within any of registers of the hardware processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a cardiac signal circuit configured to produce a sensed cardiac signal representative of electrical depolarization signals of a patient when connected to electrodes;
a heart sound sensor configured to produce a sensed heart sound signal representative of vibrational sounds of the heart;
a signal processing circuit configured to identify premature ventricular contractions (PVCs) using sensed cardiac signal information; and
a control circuit configured to:
calculate a PVC burden of the patient using PVC information;
determine that the calculated PVC burden exceeds a threshold PVC burden and that the patient is asymptomatic for heart failure;
determine that sensed heart sound signal information indicates heart failure of the patient; and
present a recommendation of an echocardiogram for the patient to a user.

2. The medical device system of claim 1, wherein the control circuit is configured to:
determine an S1 heart sound magnitude using the sensed heart sound signal information;
compare the determined S1 heart sound magnitude to a baseline S1 heart sound magnitude of the patient;
detect a decrease in the determined S1 heart sound magnitude from the baseline S1 heart sound magnitude; and
generate the recommendation of the echocardiogram in response to the calculated PVC burden exceeding the threshold PVC burden and the detected decrease in the S1 heart sound magnitude.

3. The medical device system of claim 1, wherein the control circuit is configured to:
compare the sensed heart sound signal information to a baseline heart sound signal for the patient;
detect a change in the sensed heart sound signal from the baseline heart sound signal that includes an S3 heart sound; and
generate the recommendation of the echocardiogram in response to the calculated PVC burden exceeding the threshold PVC burden and the detecting the S3 heart sound.

4. The medical device system of claim 1, wherein the control circuit is configured to:
receive information of left ventricle ejection fraction (LVEF) of the patient; and
generate the recommendation of the echocardiogram in response to the calculated PVC burden exceeding the threshold PVC burden, the LVEF information indicating normal LVEF, and sensed heart sound signal indicating heart failure.

5. The medical device system of claim 1, wherein the control circuit is configured to:
receive information of physiologic symptoms of the patient associated with heart failure; and
present an alert of heart failure status of the patient to the user when determining the patient is symptomatic for heart failure and the PVC burden exceeds the threshold PVC burden.

6. The medical device system of claim 1, including:
an ambulatory medical device (AMD) and a second device;
wherein the AMD includes the cardiac signal sensing circuit, the heart sound signal sensing circuit, and a first communication circuit configured to communicate information with the second device; and
wherein the second device includes the signal processing circuit, the control circuit, and a second communication circuit configured to communicate information with the AMD, and the control circuit is configured to upload the sensed cardiac signal information and the sensed heart sound signal information from the AMD.

7. The medical device system of claim 1, including:
an ambulatory medical device (AMD) and a second device;
wherein the AMD includes the cardiac signal sensing circuit, the heart sound signal sensing circuit, and a first communication circuit configured to communicate information with the second device; and
wherein the second device includes the signal processing circuit, the control circuit, and a second communication circuit configured to communicate information with the AMD, and the control circuit is configured to:
upload the sensed cardiac signal information from the AMD;
send a command to enable the AMD to produce the sensed heart sound signal in response to determining that the calculated PVC burden exceeds the threshold PVC burden and that the patient is asymptomatic for heart failure; and
upload the sensed heart sound signal information from the AMD.

8. The medical device system of claim 1, including:
an ambulatory medical device (AMD) and a second device;
wherein the AMD includes the cardiac signal sensing circuit, the heart sound signal sensing circuit, the signal processing circuit, and a first communication circuit configured to communicate information with the second device; and
wherein the second device includes the control circuit, and a second communication circuit configured to communicate information with the AMD, and the control circuit is configured to:
upload the PVC information and the sensed heart sound signal information from the AMD.

9. A method of operating medical device system, the method comprising:
sensing a cardiac signal of a patient using an ambulatory medical device (AMD) of the medical device system;
identifying premature ventricular contractions (PVCs) in the sensed cardiac signal;
computing a PVC burden of the patient;
determining that the patient is asymptomatic for heart failure and the computed PVC burden exceeds a threshold PVC burden;
sensing a heart sound signal of the patient using the AMD;
determining that the heart sound signal indicates heart failure of the patient; and
generating a recommendation to a user of further screening of the patient for heart failure.

10. The method of claim 9, including:
determining a baseline S1 heart sound magnitude of the patient; and
wherein the determining that the heart sounds indicate heart failure includes detecting a decrease in S1 heart sound magnitude in the sensed heart sound signal from the baseline heart sound magnitude.

11. The method of claim 9, wherein the determining that the heart sounds indicate heart failure includes detecting an S3 heart sound in the heart sound signal.

12. The method of claim 9, wherein the sensing the heart sound signal includes sensing the heart sound signal when the patient has normal left ventricle ejection fraction (LVEF) and the computed PVC burden exceeds the threshold PVC burden.

13. The method of claim 9, including:
determining the patient is symptomatic for heart failure and the PVC burden exceeds the threshold PVC burden; and
generating an alert of heart failure status of the patient.

14. The method of claim 13, wherein the determining the patient is symptomatic for heart failure includes receiving information of physiologic symptoms associated with heart failure reported for the patient.

15. The method of claim 9, including:
uploading the sensed cardiac signal from the AMD to a second device of the medical device system according to a schedule and wherein the identifying the PVCs includes the second device identifying PVCs in the uploaded cardiac signals and computing the PVC burden;
sending, by the second device, a command to the AMD to sense the heart sound signal in response to the computed PVC burden exceeding the threshold PVC burden; and
uploading the sensed heart signal to the second device, and wherein the determining that the heart sound signal indicates heart failure includes the second device determining that the heart sound signal indicates heart failure.

16. A medical device system, comprising:
a signal receiver circuit configured to receive cardiac depolarization information of a patient and heart sound information of the patient; and
a control circuit configured to:
identify premature ventricular contractions (PVCs) using the cardiac depolarization information;
calculate a PVC burden of the patient;
determine that the calculated PVC burden exceeds a threshold PVC burden and that the patient is asymptomatic for heart failure;
determine that the received heart sound information indicates heart failure of the patient; and
present a recommendation of an echocardiogram for the patient to a user.

17. The medical device system of claim 16, including:
a storage device storing a database that includes information of reported patient symptoms; and
wherein the control circuit is configured to determine that the patient is asymptomatic using the information of reported patient symptoms.

18. The medical device system of claim 17, wherein the control circuit is configured to:
determine that the patient is symptomatic for heart failure using the information of reported patient symptoms; and
present an alert of heart failure status of the patient to the user when determining the patient is symptomatic for heart failure and the PVC burden exceeds the threshold PVC burden.

19. The medical device system of claim 16, including:
a storage device storing a database that includes patient information including a baseline S1 heart sound magnitude of the patient; and
wherein the control circuit is configured to:
determine an S1 heart sound magnitude using the received heart sound information;
compare the determined S1 heart sound magnitude to the baseline S1 heart sound magnitude;
detect a decrease in the determined S1 heart sound magnitude from the baseline S1 heart sound magnitude; and
generate the recommendation of the echocardiogram in response to the calculated PVC burden exceeding the threshold PVC burden and the detected decrease in the S1 heart sound magnitude.

20. The medical device system of claim 16, including:
a storage device storing a database that includes patient information including a baseline heart sound signal for the patient; and
wherein the control circuit is configured to:
compare the received heart sound information to baseline heart sound signal;
detect a change in the received heart sound information from the baseline heart sound signal that includes an S3 heart sound; and
generate the recommendation of the echocardiogram in response to the calculated PVC burden exceeding the threshold PVC burden and the detecting the S3 heart sound.
